(19) United States
(12) Patent Application Publication
(10) Pub. No.: US 2005/0090796 A1
(43) Pub. Date: Apr. 28, 2005

(54) Disposable sanitary panty

(76) Inventors: Ischysia Coleman, Tinley Park, IL (US); Keena Kelly, Matteson, IL (US)

Correspondence Address: JoAnne M. Denison;DENISON & ASSOCS, PC, Suite 1608, 212 W. Washington Blvd , Suite 2004, Chicago, IL, 60606, US

(21) Appl. No.: 10/882,988

(22) Filed: Jun. 30, 2004

Related U.S. Application Data
(63) Continuation-in-part of application No. 10/694,204, filed on Oct. 27, 2003.

(51) Int. Cl. A61F 13/15

(52) U.S. Cl. 604/387

(57) ABSTRACT

The present invention consists of a disposable sanitary panty containing a thickened crotch pad which in one preferred embodiment has a comfortable bikini type fit which is high up on the legs and rear of the panty. It may be further provided with miniature hook and loop tabs at one or both sides of the device so that the wearer can easily put it on or take it off. It may also be provided with elastic gussets along the sides of the device for comfort and superior fit for the wearer. Further, a pocket may be provided in either the front panel, crotch area, and/or the rear panel of the device so that a gel pack which has been heretofore heated or cooled may be inserted within this pocket. Additionally, in yet an additional preferred embodiment of the present invention, the rear panel may be of a thong cut, for either additional comfort to the wearer, or to eliminate panty lines when slacks or shorts are worn. The disposable sanitary panty may be made from a paper fabric, plastic or nylon fabric, a knit type fabric, or it may be constructed from several layers, with the exterior and interior layer being made from cotton for comfort and to alleviate the possibility of skin allergies.

This application is a continuation-in-part of my co-pending application Ser. No. 10/694,204, filed Oct. 27, 2003, now abandoned.

### FIELD OF THE INVENTION

The present invention relates to the field of disposable devices used during menstruation and for postpartum purposes, and more particularly a panty that has pockets for heating or cooling pads.

### BACKGROUND OF THE INVENTION

Currently, in the marketplace, there are available a wide variety of disposable panties, and more particularly, those that might be used to hold a sanitary pod.

For example, U.S. Pat. No. 6,367,089 issued to Van Gompel, et al. discloses the use of a three dimensional disposable panty for holding a sanitary pad which has a thickened waist bond area. The sanitary pad is not integral to the device.

Further, U.S. Pat. No. 4,044,769 issued to Papajohn discloses the use of a panty which has over lopping side panels into which a sanitary pad may be retained within that includes the use of velcro tabs to retain the sanitary pad.

Similarly, in U.S. Pat. No. 6,558,364 issued to Santa Cruz, et al. discloses the use of a sanitary undergarment which is designed to be used with sanitary pods having wings. It has a crotch area with extended width to prevent soiling.

An additional patent disclosed by the prior art is U.S. Pat. No. 2,571,356 issued to C. Gemora which uses three elongate straps which attached to a waist belt in order to secure a sanitary pad to the inside portion of an undergarment.

Shown in U.S. Pat. No. 4,664,663 issued to Brier in FIG. 7 discloses a specialized sanitary pad retainer device which is used to secure within the interior of an undergarment which comprises a waterproof encasement.

All of the above are in addition to a wide variety of belts, straps and other devices which are designed primary to secure a sanitary pad to the interior of an undergarment.

However, nowhere in the prior art is seen a specialized disposable undergarment which has an integral pad and panty all in one, which further has side fastening elements to make the undergarment simple to put on or take off, even when mobility is limited, where a woman has had a Cesarian section, for example. Typically, after a woman has had a baby, the flow of blood is many times her normal period, making the assembly of sanitary pad and undergarment crucial to providing freedom from worry due to staining or leakage. In the past, during the postpartum period, in the hospital a woman was simply given a supply of extra absorbent sanitary pads and a one size fits all mesh, open panty. The pads would slip and slide around in the panty, and often leakage would occur. Staining to expensive and delicate undergarments was common. Prior approaches to this problem that hospitals took was completely inadequate and rudimentary to the problem of excessive and heavy blood flows.

### SUMMARY OF THE INVENTION

The present invention consists of disposable undergarment for women that are either menstruating, or have postpartum bleeding. The disposable undergarment is specially designed such that the pad and panty are all in one, fit comfortably, can come in different sizes, such as small, medium, large and queen sizes, and are further provided with elastic gusseting for extra comfort and proper fit. In addition, a set of velcro tabs can be provided on either one or both sides of the garment so that it can be easily put on or removed, even when a woman is quite ill after childbearing, which is not uncommon after a Cesarian section or other medical procedures which can leave the patient weak and uncomfortable.

To ensure that no leakage occurs, the undergarment may be made in layers, with a central plastic layer which would be completely waterproof, or the exterior layer may be made from a fibrous plastic or paper like material that allows the fabric to breathe, but the pores of the fabric are sufficiently small that no water molecules or larger may pass therethrough.

It is also preferred that the waist area of the garment be of a bikini type, that is, of a substantially slender nature, so that the panty is even more comfortable. In one preferred embodiment, the garment may be of a thong type, or with a simple strap in the rear of the garment, which some women find more comfortable and which can reduce or eliminate panty lines when pants are worn. A relatively wide elastic band is provided in the upper marginal portion of the garment for additional comfort during wearing of the panty.

In yet an additional preferred embodiment of the present invention, a pocket may be provided in either the front, the crotch area, or the rear of the device. After a child is born, the uterus contracts vigorously, typically resulting in a great amount of pain and discomfort to the patient. Hot or cold pads can often help reduce swelling and the contractions that occur as the uterus shrinks back into shape. For any and all discomfort resulting from child birth including, but not limited to, the aftermath of an episiotemy, a pocket may be conveniently located in the crotch area of the panty giving the opportunity to insert replocable hot or cold pads. In other instances, women can undergo “back labor”, that is where the muscles in their lower back involuntarily contract during labor, leaving the patient with severe back muscle soreness. Hence, a pocket in the rear portion of the device, may be especially helpful for such women.

In yet another preferred embodiment of the present invention, the pad is not integral to the disposable panty and a set of elastic bands is provided in the interior crotch portion of the panty which are adopted to receive a pad therebetween.

### OBJECTS OF THE INVENTION

Thus, it is one primary object of the present invention is to provide a disposable sanitary undergarment having a substantially narrow or reduced waist area, such that it produces a bikini type fit which is comfortable to the wearer.

It is still a further primary object of the present invention is to provide a disposable sanitary undergarment having a thong style back which provides increased comfort to some women and it eliminate panty lines when worn under slacks.

It is yet an additional primary object of the present invention is to provide a disposable sanitary undergarment having at least one layer of fabric that is waterproof in nature.

Yet another primary object of the present invention is to provide a disposable sanitary undergarment having at least one layer of fabric which is breathable yet waterproof, that is, air may readily pass through the fabric, yet the fabric has pores which are too small to allow water molecules to pass though.

Still an additional primary object of the present invention is to provide a disposable sanitary undergarment having elastic gussets at either side of the garment to allow it to have an improved fit and be more comfortable to the wearer.

Another primary object of the present invention is to provide at least one pocket conveniently located in either of the back side, front, or crotch area of the device in order to reduce any discomfort at the target area by exposing that area to warmth or cool via either a cold or hot gel pod.

It is still a primary object of the present invention is to provide a disposable sanitary undergarment which has miniature hook and loop fasteners on at least one side of the garment, which allows the wearer to easily put the garment on or remove it as desired.

And still yet another primary object of the present invention is to provide a disposable sanitary undergarment which may be provided with a pair of opposing elastic bonds in the crotch portion which are adapted to receive the ends of a sanitary pod.

A further primary object of the present invention is to provide a disposable sanitary undergarment which is provided with a set of opposing flexible elastic bands in the interior crotch portion of the device, wherein the opposing flexible elastic bands are adapted to removably receive and retain a standard elongate sanitary pod for the purposes of collecting bodily fluids and waste.

These and other objects and advantages of the present invention can be readily derived from the following detailed description of the drawings taken in conjunction with the accompanying drawings present herein and should be considered as within the overall scope of the invention.

### BRIEF DESCRIPTION OF THE DRAWINGS

FIG. 1 is a front perspective view of a preferred embodiment of the present invention.

FIG. 2 is a front perspective view of a second preferred embodiment of the present invention, showing a tab opened up.

FIG. 3 is a front view of a person wearing a third preferred embodiment of the present invention as a pad is being inserted into a front pocket provided on the device.

FIG. 4 is a rear view of a person wearing a third preferred embodiment of the present invention showing a thong type back panel.

FIG. 5 is a rear view of the present invention shown in FIG. 1 wherein a pad is being inserted into a rear pocket provided on the device.

FIG. 6 is a cross section taken along line A-A of FIG. 5 showing the various construction layers of the device.

FIG. 7 is a bottom perspective view of the present invention shown in FIG. 1 wherein a pad is being inserted into a crotch pocket provided on the device.

FIG. 8 is an interior plan elevation view of a crotch portion of yet an additional preferred embodiment of the present invention.

### DETAILED DESCRIPTION OF THE DRAWINGS

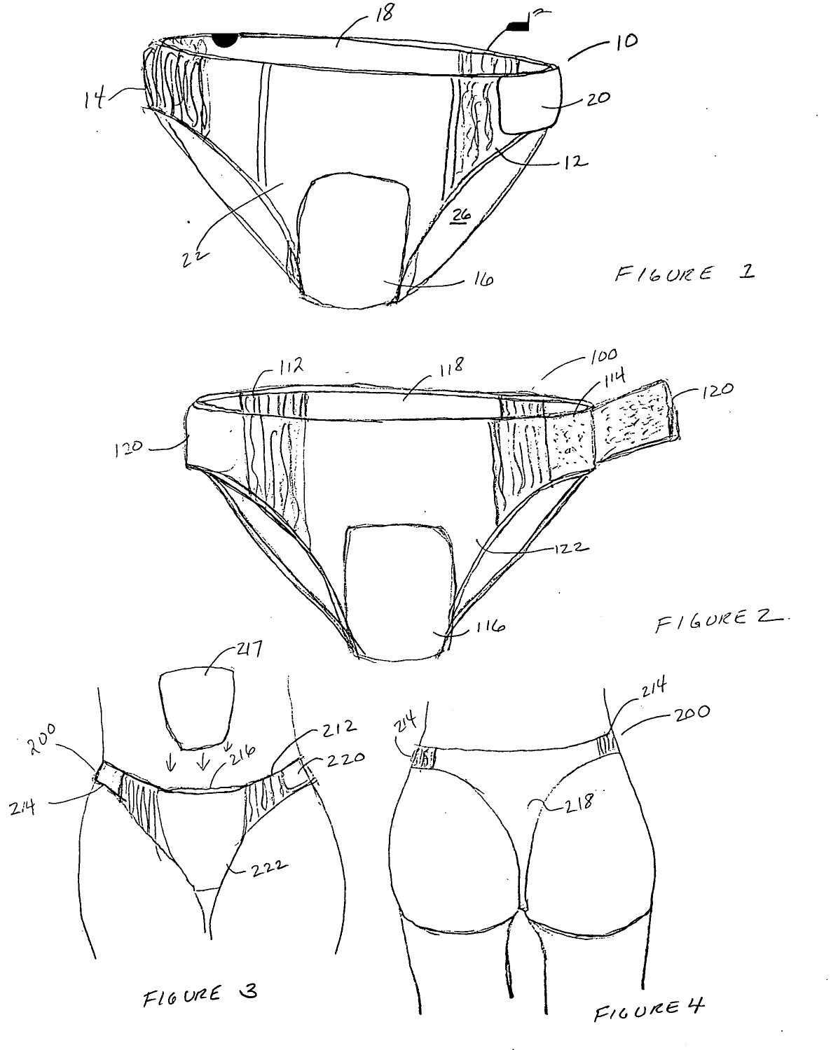

Shown in FIG. 1 is disposable sanitary panty 10 having a font panel 22, a rear panel 18, and a sanitary pad 16 traversing front panel 22 and rear panel 18. It should be noted that both front panel 22 and rear panel 18 are substantially triangular in shape, creating a bikini type cut to the device. Both the front panel 22 and the rear panel 18 are cut substantially high in the leg area and ride several inches below the wearer's waist, providing for superior comfort and fit. Disposable sanitary panty 10 is also provided with a side elastic gusset 14 along the left side of the device, whereas, on the right side of the device, two elastic panels 12 are provided in the front and rear of the device to provide superior fit and comfort. The left side of disposable sanitary panty 10 is further provided with a miniature hook/loop fastener tab 20 which the user may simply pull apart and/or push together when the device is put on or taken off. Generally, when a woman has her period, or especially during her postpartum period, the putting on or removing of undergarments, especially those that are soiled, may be more difficult than usual, and hence providing a miniature hook/loop tab 20 is especially desired by the wearer.

Of course, the front panel 22 and the rear panel 18 may be made from a wide variety of materials, including those that are breathable, yet waterproof, that is, they contain micropores which readily allow air to pass through the fabric, yet the pores are too small to allow a water molecule to pass there through.

Or, as in FIG. 1, a cotton liner 26 has been provided which allows for additional comfort to the wearer. This is especially important for wearers that have allergies, or sensitive skin that will not tolerate the chemicals present in certain papers and plastic fabrics.

Shown in FIG. 2 is disposable sanitary panty 100 which is provided with a similar front panel 122 and a rear panel 118 and a sanitary pad 116 which traverses the two elements. However, in disposable sanitary panty 100, two velcro tabs flaps 120 are provided at either side of the garment, which lie adjacent four side elastic gussets 112 for even greater ease in putting on or taking off the garment. Also shown is interior miniature hook/loop tab 114 which contains a corresponding surface of miniature hooks or loops which the interior surface of velcro tab flap 120 may readily adhere thereto.

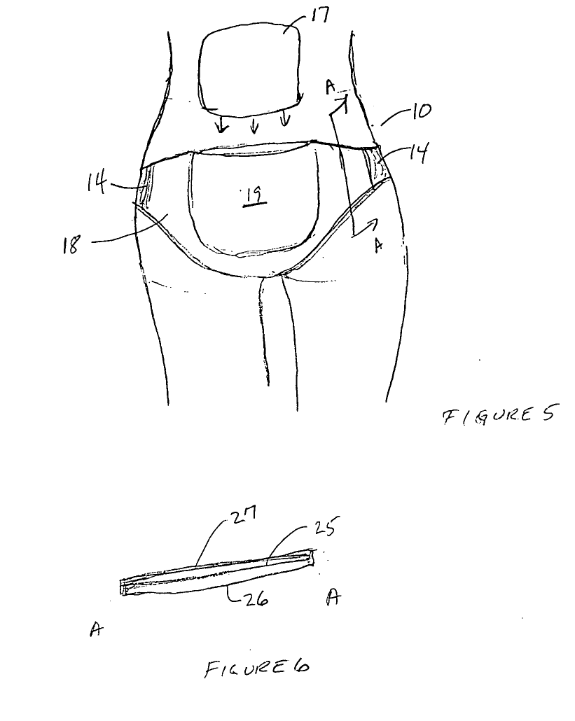

Next, shown in FIG. 3 is an additional preferred embodiment of the present invention, disposable sanitary panty 200 which is further provided with a front pocket 216 in the front panel 222 of the device. In this illustration, a packet 217 is provided which may be heated or cooled to provide relief to a wearer of the device who is in pain or discomfort. The packet 217 may be filled with gel, water or even silicone for superior heat or cold retention. As shown in the other embodiments, this disposable sanitary panty 200 is also provided with side elastic gussets 212 and 214 and a miniature hook/loop flop 220 so that the device may be easily put on or taken off.

Shown in FIG. 4 is a rear view of disposable sanitary panty 200 in which is show a back panel 218 having a substantially narrow triangular configure so as to constitute a thong type garment, which is preferred by some women in that they find it more comfortable to wear and it also eliminates panty lines from showing under slacks or shorts. Side elastic gussets 214 are also shown and are provided for comfort and fit for the wearer.

FIG. 5 shows additional features of the device shown in FIG. 1, namely disposable sanitary panty 10. In this view is shown a rear pocket 19 on back panel 18 which is adapted to receive a packet 17 which has been either heated or cooled to provide pain or discomfort relief to the wearer. Likewise, packet 17 can be filled with water, gel or silicone or a wide range of substances known in the prior art and readily available in the marketplace which provide superior heat or cold retention, and yet are soft and conformable to the wearer's body.

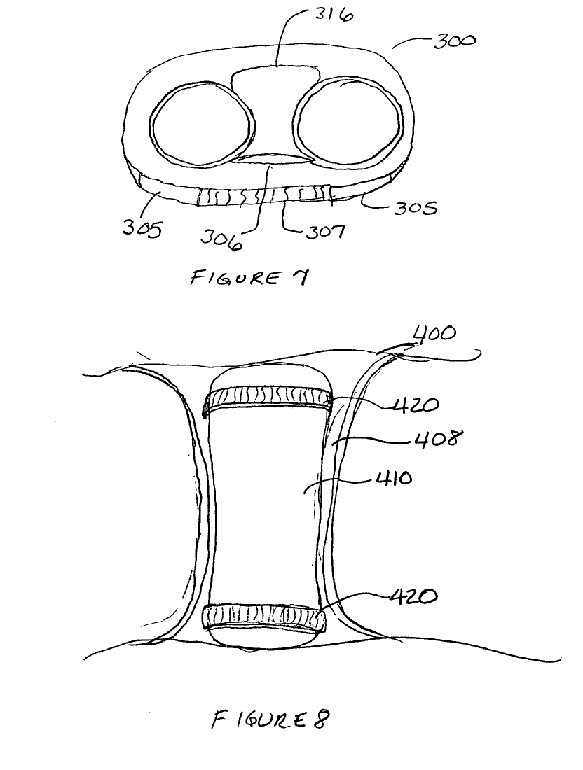

In FIG. 6 is shown a cross section elevated view taken along line A-A shown in FIG. 5. In this view three layers can be shown, first a cotton outer layer 27, then a plastic or rubber interior waterproof layer 25 and then a cotton interior lining 26. Many women find that all cotton cloth provides superior comfort and relief from allergies due to sensitive skin.

Further, FIG. 7 shows a bottom perspective view of the disposable sanitary panty 300, with opposing side velcro tabs 305 and elastic waist 307, crotch pocket 316 adapted to slidingly receive a removable gel pad, which may be either heated, or chilled, depending on the condition of the user, to relieve discomfort from trauma in the vaginal area including, but not limited to, an episiotemy. Pocket opening 306 is provided to receive the gel pad therein.

Finally, FIG. 8 shows a plan elevation view of the interior portion of yet an additional preferred embodiment of the present invention, disposable panty 400. In this particular embodiment, crotch portion 408 is provided with a set of opposing elastic bonds 420 which are secured to the interior surface of crotch portion 408. In such a manner, sanitary pod 410 is easily removably inserted into and retained by the pair of opposing elastic bands 420. The particular embodiment of the present invention 400 is desirable when the user wishes to chose from a variety of sanitary pods commonly available in the marketplace and this provides her with a wider range of options for her own personal comfort and needs. By using such a system, the wearer can choose between thin and lightweight pods for days with lesser flow, while on days where flow is very heavy, a heavier pad may be chosen.

Although in the foregoing detailed description the present invention has been described by reference to various specific embodiments, it is to be understood that modifications and alterations in the structure and arrangement of those embodiments other than those specifically set forth herein may be achieved by those skilled in the art and that such modifications and alterations are to be considered as within the overall scope of this invention.

1. A disposable sanitary panty which is provided with a front panel substantially triangular in shape, a rear panel which is substantially triangular in shape, a crotch portion extending between the front and rear panels having an interior surface, wherein both the front and rear panels are provided with substantially narrow side portions, and wherein at least one of the substantially narrow side portions is provided further with a tab having a miniature hook and loop fastening element, and the substantially narrow side portion immediately adjacent it is provided with a second tab with a corresponding hook and loop fastening element wherein the user can easily put on or remove the device.

2. The disposable sanitary panty according to claim 1 wherein both side portions of the device are provided with tabs having miniature hook and loop fastening elements on both the front and rear panels of the device.

3. The disposable sanitary panty according to claim 1 wherein at least a portion of the front and rear panels of the device are made from a breathable material which resists moisture from passing there through.

4. The disposable sanitary panty according to claim 1 wherein both the front and rear panels of the device are provided with at least one elastic gusset portion for improved comfort and fit to the wearer of the device.

5. The disposable sanitary panty according to claim 1 wherein the back panel of the device has a triangular configuration that is substantially narrow as it runs vertically from top to bottom, providing for a thong like appearance.

6. The disposable sanitary panty according to claim 1 wherein the front panel of the device is provided with a pocket.

7. The disposable sanitary panty according to claim 1 wherein the rear panel of the device is provided with a pocket.

8. The disposable sanitary panty according to claim 1 wherein the crotch area of the device is provided with a pocket.

9. The disposable sanitary panty according to claim 6 wherein the device is further provided with a packet filled with a liquid substance having a configuration which is adapted to fit into the pocket provided in the front panel of the device.

10. The disposable sanitary panty according to claim 7 wherein the device is further provided with a packet filled with a liquid substance having a configuration which is adapted to fit into the pocket provided in the rear panel of the device.

11. The disposable sanitary panty according to claim 8 wherein the device is further provided with a packet filled with a liquid substance having a configuration which is adapted to fit into the pocket provided in the rear panel of the device.

12. The disposable sanitary panty according to claim 1 further having a cotton inner layer to provide comfort to the wearer of the device.

13. The disposable sanitary panty according to claim 1 further having a cotton exterior layer to provided comfort to the wearer of the device.

14. The disposable sanitary panty according to claim 1 further having a waterproof layer to prevent bodily fluids from leaking outside the device.

15. The disposable sanitary panty according to claim 1 further having an integral absorbent pad adjacent the crotch portion of the device.

16. The disposable sanitary panty according to claim 1 further having an elongate absorbent pad adjacent the crotch portion of the device.

17. The disposable sanitary panty according to claim 1 where in at least two flexible, deformable elongate band members are provided in the crotch portion of the device which are adapted to receive and removably retain the absorbent pad.
